(19) United States
(12) Patent Application Publication
(10) Pub. No.: US 2023/0165971 A1
(43) Pub. Date: Jun. 1, 2023

(54) Transfection and Transduction System

(71) Applicant: N4 Pharma UK Limited, Derby (GB)

(72) Inventors: Michael Themis, Aylesbury (GB); David Templeton, Welwyn Garden City (GB)

(21) Appl. No.: 17/920,317

(22) PCT Filed: Apr. 21, 2021
(86) PCT No.: PCT/GB2021/050960

§ 371 Date: Oct. 20, 2022

(30) Foreign Application Priority Data
Apr. 23, 2020 (GB) 2005957.2

(51) Int. Cl. A61K 47/69; A61K 47/59

(52) U.S. Cl. CPC A61K 47/6923; A61K 47/59; A61K 47/6901

(57) ABSTRACT

The invention relates to the fabrication and use of silica organic nanoparticles as delivery vehicles for vims and virus-like species to the body. The nanoparticles typically have a hollow core and a surface morphology that allows effective adhesion of species to the surface for delivery to the body. In particular, the invention is particularly useful for performing transfection and transduction.

### FIELD OF INVENTION

The invention relates to a composition comprising a nanoparticle and one or more delivery components, and methods of introducing the delivery components to a cell. The invention also relates to the use of the composition in therapy.

### BACKGROUND

Gene therapy has reached the clinic following substantial pharmaceutical investment. Applications for clinical trials are also increasing with over 1800 approved between 2015 and 2017 using virus-based products such as Stremvellis (GSK), Glybera (Uniqure) and Kymriah (Novartis) that deliver therapeutic genes to correct genetic disease in humans. Gene therapy relies on these vectors to deliver functional copies of therapeutic genes, however, a huge limitation facing successful gene therapy is the large scale production of these vectors within a reasonable budget for the clinic. An example of this limitation is the withdrawal of Glybera, a therapy that was destined to treat lipoprotein lipase deficiency. The overall cost of this therapy was determined to be too high owing to a number of factors including the orphan indication, the research and development costs and the production costs.

Over the past 40 years, several protocols for virus vector production have been developed for gene therapy, and costs are reducing. Vectors are usually generated as replication-incapable viruses for safety reasons. To make these vectors, the genes that are usually carried on the wild type virus genome needed for their production have been removed. These genes, including genes for the virus structure and envelope (required for cell attachment), are placed on plasmid vectors. The plasmid vectors are then transfected into specialised producer cells to enable them to make a recombinant form of the virus that can infect but not replicate. Hence, producer cells can package only the product coded by the plasmid for the therapeutic recombinant virus genome, which has a specialised packaging signal for its selection. An example is retrovirus vectors that have their structural gag, pol and env genes removed from their genome and placed on two plasmids. A third plasmid carries the therapeutic genome and all three plasmids are transfected into human cells to make the therapeutic infectious replication deficient retrovirus vectors.

A major drawback is that certain envelopes are toxic to the producer cells and hence virus production is transient. This transient transfection is now the ‘gold standard’ way used to make retroviruses, adenoviruses and even adeno-associated viruses. For several years, research groups have searched for the optimal transfection agent to transfect cells. Some examples of commonly used transfection agents are polyethylenimine (PEI), FuGENE®, Lipofectamine and Transfectam™.

Ideally, a transfection reagent that can bind all three plasmids at once would have the best chance of all three plasmids reaching the same cell for good virus production.

### SUMMARY OF INVENTION

There is provided in a first aspect of the invention a composition comprising: an inorganic nanoparticle; and one or more delivery components; wherein the nanoparticle comprises projections thereon; wherein the nanoparticle has a diameter in the range 50 nm to 3000 nm; wherein the inorganic nanoparticle is at least partially coated with a transfection agent; and wherein the one or more delivery components are selected from: vectors, viral vectors, plasmids, viruses, viroids, prions, virus-like particles, virus-derived components and mixtures thereof.

The composition is suitable for performing transfection and transduction.

The term “nanoparticle” is intended to take the usual meaning in the art. That is to say, it encompasses particles having an average diameter on the nanoscale, i.e. in the range of 1 nm to 1000 nm, when the particles are approximated to spheres.

The term “diameter” as used herein is intended to describe the diameter of the nanoparticle when approximated to a sphere, including the projections provided on the nanoparticle surface.

For the avoidance of doubt, the term “inorganic” is intended to refer to materials which do not comprise carbon.

Typically, the nanoparticles comprise an inorganic material selected from: silica, titania, alumina or a combination thereof. However, in many instances, the nanoparticles will comprise silica. It has been found that silica functions well as a nanoparticle in the present invention as it can be readily functionalised as needed with an array of different species. The nanoparticles may consist of, or consist essentially of, the inorganic material. A composition which consists essentially of a specified component includes the specified component wherein any other component present is provided in an essentially negligible amount (for instance, less than 0.5 wt %) i.e. any addition ingredient or ingredients do not materially affect the function of the specified components.

The nanoparticles are typically hollow. Whilst many delivery systems make use of a vesicle or micellar structure to internalise a delivery component and then subsequently eject said component on arrival at a target site, the hollow nanoparticles do not typically contain delivery components within their hollow structure. However, in some embodiments, delivery components or other materials useful in bringing about transfection, may be carried within the hollow structure of the nanoparticles. Other components which are not useful in bringing about transfection, for example pharmaceutically active agents, may be carried within the hollow structure of the nanoparticles, alone or together with other components or delivery components. The components may be released to act separately or together with the effect caused by transfection.

In some embodiments, the nanoparticles may comprise: a shell comprising an inorganic compound; a hollow core with a volume defined by the inner surface of the shell; and a plurality of projections comprising the inorganic compound disposed on the exterior of the shell. Typically, the projections are integral with the shell.

The term “mesoporous” is intended to take its normal meaning in the art. In particular, it refers to a material comprising mesopores, i.e. pores having widths (i.e. pore sizes) of from 2 nm to 50 nm, more typically 5 nm to 40 nm and in some instances 10 nm to 30 nm. Said pores are typically located in the shell of the particle and typically extend through the entire thickness of the shell. Material can in some embodiments be loaded into the hollow core of the nanoparticles via the pores. However, in many instances the core remains empty, i.e. no materials are specifically introduced into the core.

Typically, the nanoparticle is at least partially coated with the one or more delivery components. The delivery components are typically coated onto the external surface of the nanoparticles. However, the nanoparticle may be functionalised with one or more functional groups in order to encourage the bind of delivery agents.

The inorganic nanoparticle is at least partially coated on its external surface with a transfection agent. The term “transfection agent” is intended to take the usual meaning in the art. In particular, it refers to a compound or substance which enhances the ability of active agents to be transfected. This could be by enhancing the ability of either the delivery system itself (in the present cases, the inorganic nanoparticle) or the active material to penetrate the cell membrane; and/or by improving the ability of the active ingredients to become incorporated into the target cell's genetic material. In the present case, the transfection agent is typically provided to mask the charge on the inorganic material to enhance the binding of delivery agents to the nanoparticle.

The combination of transfection agents together with the inorganic nanoparticle described herein has been found to be especially effective at promoting transfection.

Often, the transfection agent will substantially coat at least half of the surface of the nanoparticles, more typically at least 75% of the surface of the particles and, most typically, substantially all of the surface of the nanoparticles. The term “substantially all” as used herein typically means greater than or equal to 95% of the surface of the nanoparticle.

It is often the case that the transfection agent is a polymer. Polymeric materials provide a good coating of the particles. Moreover, the mechanism of transfection agents often uses a polymeric structure in order to maximise their effectiveness. The polymer may be a copolymer. For example, the copolymer may be: a block copolymer, alternating copolymer, statistical copolymer, or combination thereof.

The choice of transfection agent is not particularly limited. Typically, the transfection agent is cationic. Cationic compounds, and especially cationic polymers, are advantageous as they encourage the binding of negatively charged species such as a nucleic acid. The cationic polymer typically comprises a polyamine. The cationic polymer may be a polypeptide, for instance polyarginine, polylysine or polyhistidine.

In some embodiments, the cationic compound is chitosan or a derivative thereof. In some instances, a proportion of the amino groups in the chitosan are alkylated, in many cases trialkylated (i.e. alkylated with three alkyl groups, for instance with three methyl groups). Alternatively, the cationic polymer may be a polyamidoamine (PAMAM), a PAMAM dendrimer, a polylysine, a DEAR-dextran or polybrene. Commercial transfection agents may also be used such as FuGENE®, Lipofectamine and Transfectam®.

It may be the case that the transfection agent comprises a polyalklylimine. As one skilled in the art would appreciate, it is advantageous in some situations for the transfection agent to include a series of anime linkages (—NH—). Such groups have been shown to react with nucleic acids in a manner which enhances delivery to a cell. Furthermore, the transfection agent for use in the present invention may be a combination of two or more of those described above.

There is no particular limitation on the type of polyalkylimine employed, it may be: a linear, branched, or dendritic polyalkylimine and may be a combination thereof. Often a branched or dendritic polyalkylimine will be employed. The polyalkylimine may have a weight average molecular weight in the range of 2,000 da to 40,000 da, more typically in the range of 10,000 da to 25,000 da. In some embodiments, the polyalkylimine has a weight average molecular weight in the range of 3,000 da to 7,000 da, more typically about 5,000 da.

Typically, the nanoparticles comprise at least 1.0% by weight of the transfection agent. More typically, the nanoparticles comprise at least 2.0% by weight or in some instances at least 5.0% by weight of transfection agent. Typically, the nanoparticles comprise in the range of 6.0 to 15% by weight of the transfection agent.

Typically, the polyalkylimine will be a polyethylenimine (PEI). PEI has been shown to work well as a transfection agent. PEI may be linear or branched. Moreover, it is common for the PEI to be a branched PEI.

In addition, it is typically the case that the nanoparticles further comprise one or more moieties for binding the transfection agent thereto. As will be appreciated by one skilled in the art, different transfection agents will bind differently to nanoparticles fabricated from different inorganic materials. As such, a range of moieties can be employed to ensure a good bond between the nanoparticle and the transfection agent. However, it is typically the case that the outer surface of the nanoparticle, is at least partially coated with one or more acidic groups. Typical examples of acidic groups include, but are not limited to: phosphonate, phosphate, sulfate, carboxylate, alpha-keto carboxylate, or combinations thereof. Of these groups, phosphate, phosphonate and sulfate are most frequently used. Typically, the acid group is a phosphonate, such as a methylphosphonate (e.g. 3-(Trihydroxysilyl)propyl methylphosphonate). Providing acidic groups on the surface of the nanoparticle is advantageous as these groups are typically negatively charged. This increases the negative on the charge surface of the nanoparticle which in turns improves the binding of the transfection agent to the nanoparticle surface.

The core, i.e. the void within the shell, typically has a diameter in the range of 50 nm to 500 nm, for instance from 75 to 300 nm. Further, the shell typically has a thickness in the range of 10 nm to 200 nm.

The particle size of the nanoparticles is typically from 50 nm to 3000 nm, more typically 75 nm to 2000 nm, even more typically 100 nm to 1000 nm and often in the range 100 nm to 500 nm. In some instances, the nanoparticles in the range of 100 nm to 300 nm. For the avoidance of doubt, the term “particle size” herein is intended to describe the diameter of a nanoparticle when approximating the particle to a sphere. Moreover, the average particle size of a plurality of nanoparticles is typically 50 nm to 3000 nm, more typically 75 nm to 2000 nm, even more typically 100 nm to 1000 nm and often in the range 100 nm to 500 nm. In some instances, the nanoparticles in the range of 100 nm to 300 nm. The term “average” is intended to refer to a mean value. The particle sizes referred to herein are typically as measured using dynamic light scattering or by reference to SEM images.

The nanoparticles typically have a rough or “spiky” surface morphology. For example, the nanoparticles may be rambutan-like or morningstar-like. In particular, the projections on the surface of the particles often form a plurality of spikes or finger-like structures on the surface between which matter can become enmeshed.

The projections, which extend generally radially outwards from the shell, typically the same inorganic material as the shell. The projections typically increase the surface area of the hollow nanoparticle. Typically, the projections have a length equal to or less than the diameter of the core. More often, the length of the projections is in the range of 5 nm to 1000 nm, more typically in the range 10 nm to 200 nm, and even more often in the range of 50 nm to 150 nm. Whilst the length of the projections is typically substantially uniform, there may be variation in the length of the projections. The term “substantially uniform” as used herein typically means±15% from the mean average protrusion length.

The projections typically have a diameter in the range of 2 nm to 50 nm, more typically 5 nm to 25 nm, and even more typically around 10 nm to 20 nm. The diameter referred to herein refers to the diameter of the projections at their bases i.e. where the projections abut the shell.

The nanoparticles may be highly monodisperse. Typically, the polydispersity index (PDI, also known as the dispersity index) of the nanoparticles is less than or equal to 0.3, more typically less than or equal to 0.15, even more typically less than or equal to 0.1, and in some cases less than or equal to 0.05. The dispersity index can be calculated as the ratio of the quadratic average (i.e. the average value of squares of measured diameters, d), and square of arithmetic average of measured diameters. The calculations for the dispersity index may be as defined in the ISO standard document 13321:1996 E and ISO 22412:2008.

The nanoparticles typically have high surface areas. For instance, the nanoparticles may have a BET surface area of at least 120 cm²/g, or more typically instance at least 150 cm²/g. In some embodiments, the nanoparticles have a BET surface area of at least 140 cm²/g. The plurality of hollow nanoparticles may have a mean particle size in the range of 160 to 250 nm and a BET surface area of at least 120 cm²/g. The BET surface area may for instance be measured using the ISO 9277 standard. The BET surface area may also be measured based on adsorption and desorption of nitrogen.

The nanoparticles typically comprise at least 70% by weight of the inorganic material relative to the total weight of the nanoparticle. In some instances, the inorganic material may comprise at least 90% by weight of the nanoparticle, or more typically at least 95% by weight of the nanoparticle.

Without being bound by theory, the inventors have found that nanoparticles comprising projections, or “spiky” nanoparticles, improve the efficiency of transfection and transduction.

The term “delivery component” is intended to refer a species which is introduced to a cell in order to effect, or assist, transfection or transduction.

The one or more delivery components are typically attached to the external surface of the nanoparticles. Without being bound by theory, it is believed that the “spiky” structure is able to effectively enmesh delivery components and facilitate their conveyance to a target cell to effect transfection or transduction.

It is often the case that the composition comprises at least two delivery components and in some cases at least three delivery components. The nanoparticles of the invention have been found to be capable of carrying multiple different delivery components on a single nanoparticle. This is particularly useful as it allows for combination therapies to be delivery simultaneously. For example, the inventors have demonstrated that a plurality of delivery components can be complexed to the spiky nanoparticle and delivered to a cell.

In some embodiments, the nanoparticle may be at least partially coated with the one or more delivery components. The nanoparticle may be at least 10%, 20%, 30%, 40%, 50%, 60%, 70%, 80% or 90% coated with the one or more delivery components. The nanoparticle may be fully coated with the one or more delivery components (that is to say equal to or 95% coated).

The amount delivery component with respect to the nanoparticle will vary based on the choice of delivery component and the dosage required for a given application. However, typically, the weight ratio of delivery component to the nanoparticle is in the range of 1:100 to 100:1, more typically 1:50 to 5:1, even more typically 1:30 to 1:1 and most typically 1:20 to 1:2.

Typically the one or more delivery components are independently selected from vectors; viruses; viroids; prions; virus-like particles; virus-derived components and mixtures thereof. Typical vectors include viral vectors and plasmids.

The term “vector”, as used herein, is intended to refer to a nucleic acid molecule capable of transporting another nucleic acid to which it has been linked. Vectors are well known in the art and any type of vector may be used. One type of vector is a “plasmid”, which refers to a circular double stranded DNA into which additional nucleic acid segments may be ligated. Another type of vector is a viral vector, wherein additional nucleic acid segments may be ligated into the viral genome. Another type of vector is a cosmid. Another type of vector is an artificial chromosome, including yeast artificial chromosomes (YACs), bacterial artificial chromosomes (BACs) and human artificial chromosomes (HACs).

Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. In general, expression vectors of utility in recombinant DNA techniques are often in the form of plasmids. Certain vectors used in accordance with the practice of invention described herein may be well-known vectors used in the art, such as plasmids derived from pBR322, pUC, pCMV, pMDG, pHR and mixtures thereof or viral vectors derived from retroviruses, adenoviruses or adeneo-associated viruses and mixtures thereof.

Non-limiting examples of the types of modification to a vector that may be suitable in the practice of the present invention include, though are not limited to, modification such as the addition of modification of one or more enhancers, one or more promoters, one or more ribosomal binding sites, one or more origins of replication, or the like. In certain non-limiting embodiments, an expression vector used in the practice of the present invention may include one or more enhancer elements selected to improve expression of the protein of interest in the present transient expression system. The selected enhancer element may be positioned 5 ‘ or 3’ to the expressible nucleic acid sequence used to express the protein of interest.

Preferably, the one or more delivery components comprises a vector. More preferably, the one or more delivery components comprises a viral vector, preferably a lentiviral vector, a plasmid, preferably derived from pCMV, pMDG or pHR or a mixture thereof.

The vector may be empty or comprising an insert or transgene. The insert or transgene may comprise exogenous DNA, RNA, small interfering RNA, microRNA, small hairpin RNA. Preferably, the insert or transgene comprises DNA.

The delivery component may be a virus. Typical viruses include, but are not limited to: retrovirus including lentivirus, adenovirus, adeno-associated virus (AAV), herpes simplex virus, or combinations thereof. Preferably the virus is a lentivirus.

Alternatively, the delivery component may be a viroid. Typical viroids include members of Pospiviroidae and Avsunviroidae.

In some embodiments, the delivery component is a prion. Typical prions include those derived from PrP.

In some embodiments, the one or more delivery components may comprise a virus-derived component, said virus-derived component selected from a virion, a capsid, a viral nucleic acid, a viral DNA, a viral RNA, or a viral protein.

In various embodiments, the virus-derived component may be made from a DNA virus or an RNA virus. The virus-derived component may be from an adenovirus, an adeno-associated virus, a herpes simplex virus, a retrovirus including a lentivirus, an alphavirus, a flavivirus, a rhabdovirus, a measles virus, a Newcastle disease virus, a poxvirus, or a picornavirus. Preferably, the virus-derived component is from a lentivirus.

In some embodiments, the one or more delivery components may comprise a virus-like particle (VLP), closely resembling a virus but comprising no viral genetic material. VLPs may be produced from components of a wide variety of virus families including Parvoviridae (e.g. adeno-associated virus), Retroviridae (e.g. HIV), Flaviviridae (e.g. Hepatitis C virus), Paramyxoviridae (e.g. Nipah) and bacteriophages (e.g. Qβ, AP205).

In some embodiments, the composition may further comprise a cell. The cell may be a eukaryotic cell or a prokaryotic cell. Preferably, the cell is a eukaryotic cell. The cell may be a primary cell or a cell derived from a cell line, including immortalised cell lines and transformed cell lines. The cell may be a plant cell or an animal cell. Preferably, the cell is a mammalian cell. More preferably, the cell is a human cell.

The cell can be any type of cell. Preferably, the cell is a leukocyte. More preferably, the cell is an antigen presenting cell or a lymphocyte, such as a B cell or a T cell. Even more preferably, the cell is a T cell. The cell may be a CD4+ T cell or a CD8+ T cell. The cell may be a helper T cell, a cytotoxic T cell or a regulatory T cell. Alternatively, the cell may be a stem cell, such as an induced pluripotent stem cell (iPSC).

The cell may be a genetically engineered cell or transgenic cell. For example, the cell may be a chimeric antigen receptor T cell (CAR-T cell) or a T cell receptor (TCR)-engineered T cell (TCR-T cell). The cell may be have been genetically engineered by any method known in the art, including but not limited to viral vectors, liposomes and electroporation. The cell may have been genetically engineered by the method described below.

The composition may additionally comprise one or more pharmaceutically acceptable excipients. The one or more excipients include carriers, diluents and/or other medicinal agents, pharmaceutical agents or adjuvants, etc.

The term “transfection” is used herein to mean the delivery of exogenous nucleic acid, protein or other macromolecule to a target cell by non-viral means, such that the exogenous nucleic acid, protein or other macromolecule is expressed or has a biological function in the cell. Non-limiting examples of exogenous nucleic acid include DNA, RNA, siRNA, miRNA, shRNA, mRNA and mixtures thereof. Typically, the exogenous nucleic acid is DNA, more typically supercoiled plasmid DNA. Transfection of the cell may be transient, i.e. the exogenous nucleic acid exists in the cell for a limited period of time and it not integrated into the target cell genome. Alternatively, transfection of the cell may be stable, i.e. the exogenous nucleic acid is either integrated into the target cell genome or maintained as an episomal plasmid, resulting in long-term maintenance of the exogenous nucleic acid in the target cell and its progeny.

The term “transduction” is used herein to mean the delivery of exogenous nucleic acid to a target cell by a virus or viral vector, such that the exogenous nucleic acid is expressed or has a biological function in the cell. Non-limiting examples of exogenous nucleic acid include DNA, RNA, siRNA, miRNA, shRNA, mRNA and mixtures thereof. Typically, the exogenous nucleic acid is DNA, more typically supercoiled plasmid DNA. Transfection of the cell may be transient, i.e. the exogenous nucleic acid exists in the cell for a limited period of time and it not integrated into the target cell genome. Alternatively, transfection of the cell may be stable, i.e. the exogenous nucleic acid is either integrated into the target cell genome or maintained as an episomal plasmid, resulting in long-term maintenance of the exogenous nucleic acid in the target cell and its progeny.

The transfection or transduction may occur in vitro or ex vivo. For example, cell line cells or isolated primary cells in culture may be transfected or transduced. Preferably, transfection or transduction may occur to a cell isolated from a patient.

There is also provided in a second aspect of the invention, a composition according to the first aspect of the invention for use in therapy. The inventors have found that the composition of the first aspect of the invention is surprisingly effective at transfecting or transducing cells. Accordingly, there is no particular limitation as to which cells may be transfected or transduced. It is envisaged that a range of different cells could be treated with a range of different delivery components in order to bring about a change in the genetic make-up of a cell. Accordingly, the therapy is typically gene therapy. Both the administration of a cell, treated according to the invention, or in vivo transfection or transduction using the composition of the first aspect of the invention can be used to treat or prevent a wide range of disorders. That said, typical disorders that can be treated or prevented include, but are not limited to: genetic disorders, cancer, infection or autoimmune disease. Typical genetic disorders include: cystic fibrosis, heart disease, diabetes, hemophilia, and retinitis pigmentosa.

The cancer may be any cancer, including haematological cancers, leukaemias, lymphomas and multiple myeloma, as well as solid tumours and other non-blood and non-haematological cancers. The infection may be any infection caused by a pathogen, including infections caused by viruses, bacteria, fungi, parasites and prions. The autoimmune disease may be any autoimmune disease, including type 1 diabetes mellitus, rheumatoid arthritis, psoriasis, psoriatic arthritis, multiple sclerosis, systemic lupus erythematosus, inflammatory bowel disease, Addison's disease, Graves' disease, Sjögren's syndrome, Hashimoto's thyroiditis, myasthenia gravis, autoimmune vasculitis, pernicious anemia, pemphigus vulgaris and celiac disease.

Typically the disease is cancer, infection or autoimmune disease.

When the disease is “treated” in the above use or method, this means that one or more symptoms of the disease are ameliorated. It does not mean that the symptoms are completely remedied so that they are no longer present in the patient, although in some methods, this may be the case. Treating the disease results in one or more of the symptoms of the disease being less severe than before treatment. Treatment may result in a plurality of the symptoms of the disease being less severe than before treatment. Further, the invention can be employed to prevent the development of a disease through prophylactic application.

There is also provided in a third aspect of the invention the use of the composition according to the first aspect of the invention in the manufacture of a medicament for therapy. The therapy is typically as described with respect to the second aspect of the invention. Again, as per the second aspect of the invention, the method includes in vivo transfection or transduction as well the delivery of cells transfected or transduced using the composition according to the first aspect of the invention.

There is also provided in a fourth aspect of the invention a method of treating a disease in a patient, comprising administering the composition according to the first aspect of the invention to a patient. Preferably, the patient is human. Preferably, a therapeutically effective amount of the composition is administered to the patient. A “therapeutically effective amount” refers to an amount effective, at dosages and for periods of time necessary, to achieve the desired therapeutic result. Moreover, said treatment is also intended to cover the delivery of a cell to a patient, wherein said cell has been transfected or transduced using the composition according to the first aspect of the invention.

There is also provided in a fifth aspect of the invention a method of manufacturing the composition according to the first aspect of the invention, the method comprising the step of: mixing the nanoparticle as described in the first aspect of the invention, with the one or more delivery components as described in the first aspect of the invention.

The nanoparticles of the invention can be fabricated using a Stober-like process using a method such as that described in on pages 24 to 28 of WO2019/097226. Where a transfection agent is applied, a further step is performed following the provision of the inorganic nanoparticles in order to coat the nanoparticles. In these situations, the inorganic nanoparticles are typically first functionalised with a linker moiety to aid the coating of the nanoparticle with the transfection agent. The linker is not particularly limited as explained above but it is often a phosphonate. Typically, the inorganic nanoparticles a mixed with the linker and stirred for at least 5 minutes (usually 30 minutes to 10 h, most often 1 h to 4 h), often at an elevated temperature above room temperature, typically in the range of 20° C. to 70° C.

In some embodiments, the mixing step may last less than 60 minutes. Typically, the mixing step lasts in the range of 10 minutes to 45 minutes, more typically 10 minutes to 30 minutes. The process is typically conducted at room temperature i.e. in the range of 15° C. to 35° C. and most typically in a polar solvent, usually water. A buffer solution may often be employed, such as a phosphate buffered saline solution.

There is also provided in a sixth aspect of the invention a method of transducing or transfecting a cell using the composition according to the first aspect of the invention. Said method comprises the steps of: (i) providing a composition according to any of claims 1 to 14; and (ii) incubating the composition with a cell.

In one embodiment, there is provided a method of transducing a cell, wherein the one or more delivery components comprise viral vectors. In particular, there is provided a method of transducing a cell comprising: (i) providing a nanoparticle having projections thereon, and wherein the diameter of the nanoparticle ranges from 100 nm to 3000 nm; (ii) providing one or more delivery components, said delivery components being viral vectors; and (iii) incubating the nanoparticle and the one or more delivery components with a cell.

In a further embodiment, there is provided a method of transfecting a cell, wherein the one or more delivery components comprise plasmids. In particular there is provided a method of transfecting a cell, the method comprising: (i) providing a nanoparticle having projections thereon, and wherein the diameter of the nanoparticle ranges from 100 nm to 3000 nm; (ii) providing one or more delivery components, said delivery components being plasmids; and (iii) incubating the nanoparticle and the one or more delivery components with a cell.

With respect to the above transfecting and transducing embodiments, typically the incubation step lasts for less than 48 h, more typically less than 24 h, and even more typically less than 5 h. Often, the incubation step lasts less than 3 h and even more typically less than 2 h. Often, the incubation step will last in the range of 5 minutes to 1 h, usually 10 minutes to 30 minutes.

As explained above, the nanoparticle may comprise a transfection agent to aid the transfection of transduction process.

Both methods can be performed in vivo or ex vivo. Typically, the method is performed ex vivo.

A skilled person will appreciate that all aspects of the invention, whether they relate to, for example, the composition, its use, or a method of treatment etc., are equally applicable to all other aspects of the invention. In particular, aspects of the composition for example, may have been described in greater detail than in other aspects of the invention, for example, the use. However, the skilled person will appreciate where more detailed information has been given for a particular aspect of the invention, this information is generally equally applicable to other aspects of the invention.

All patent and literature references cited in the present specification are hereby incorporated by reference in their entirety.

### DESCRIPTION OF FIGURES

The invention will now be described in detail by way of example only with reference to the figures which are as follows:

FIG. 1. Cell viability after 4 h treatment with Nuvec®®. Viability of cells treated with different concentrations of Nuvec®® (Negative Control (NC), 1 μg, 10 μg, 40 μg, 60 μg, 80 μg) for 4 h. After 4 h, the medium was changed. Cell survival and recovery was recorded for 72 h post-medium change, at the time points shown.

FIG. 2. Cell viability after 4 h treatment with Nuvec®®. (A, B) Cell survival over time (every 24 h) of HEK293T cells treated with a range of concentrations of Nuvec® for 4 h (n=1). See Table 3 below.

FIG. 3. Cell viability after 24 h treatment with Nuvec®. Viability of cells treated with different concentrations of Nuvec® (Negative Control (NC), 1 μg, 10 μg, 40 μg, 60 μg, 80 μg) for 24 h. After 24 h, the medium was changed. Cell survival and recovery was recorded for 72 h post-medium change, at the time points shown.

FIG. 4. Cell viability after 24 h treatment with Nuvec®. (A, B) Cell survival over time of HEK293T cells treated with a range of concentrations of Nuvec® for 24 h. See Table 4 below.

FIG. 5. Cell viability after 48 h treatment with Nuvec®. Viability of cells treated with different concentrations of Nuvec® (Negative Control (NC), 1 μg, 10 μg, 40 μg, 60 μg, 80 μg) for 48 h. After 48 h, the medium was changed. Cell survival and recovery was recorded for 72 h post-medium change, at the time points shown.

FIG. 6. Cell viability after 48 h treatment with Nuvec®. (A, B) Cell survival over time of HEK293T cells treated with a range of concentrations of Nuvec® for 48 h.

FIG. 7. Cell viability and GFP expression after 4 h, 24 h and 48 h transfection with 1 μg Nuvec® and 5 μg GFP vector only. Analysis at 72 h post-transfection.

FIG. 8. Cell viability and GFP expression after 4 h, 24 h and 48 h transfection with 10 μg Nuvec® and 5 μg GFP vector only. Analysis at 72 h post-transfection.

FIG. 9. Cell viability and GFP expression after 4 h, 24 h and 48 h transfection with 40 μg Nuvec® and 5 μg GFP vector only. Analysis at 72 h post-transfection.

FIG. 10. Cell viability and GFP expression after 4 h, 24 h and 48 h transfection with 60 μg Nuvec® and 5 μg GFP vector only. Analysis at 72 h post-transfection.

FIG. 11. Cell viability and GFP expression after 4 h, 24 h and 48 h transfection with 80 μg Nuvec® and 5 μg GFP vector only. Analysis at 72 h post-transfection.

FIG. 12. Viruses produced and titrated against indicator cells to use later to test coupling with Nuvec®. High titre LV was produced using traditional production methods and titrated using various dilutions of LV. Analysis via flow cytometry determined percentage expression of GFP at various dilutions which was used to calculate a titre of 1.18×10⁹TU/ml. LV was produced for future use with Nuvec® for transduction assays.

FIG. 13. HEK293T indicator cells were transfected using NV00100028, NV00100026-28, NV0010032 and NV0010033 with 5 μg of plasmid DNA; GFP, GAG-POL and VSV-G at a ratio of 4:3:1, for either 4 h or 24 h. Viral supernatant was harvested every 24 h for 72 h. Photos show transfected cells 72 h post-transfection.

FIG. 14. HEK293T cells transduced with viral supernatant collected of cells transfected using Nuvec® at different concentrations and transfection times. Percentage of GFP positive cells were analysed using flow cytometry 72 h post transduction and are shown below each image. Initially, it was unknown how much lentivirus Nuvec® could actually generate and 500p1 and 200p1 of viral supernatant was used to transduce HEK293T cells. For a more true representation of viral titres the calculation for lentivirus titres use percentages between 1-30% GFP positive. As shown above even at the lowest dilution used (200 p1) cells were still more than 30% GFP+ so repeats using smaller volumes was carried out.

FIG. 15. Repeat titrations were carried out using smaller volumes of viral supernatant collected of cells transfected with different concentrations of Nuvec®. HEK293T cells were then transduced with 100p1 and 10p1 of viral supernatant and percentage of positive green cells was determined using flow cytometry 72 h post-transduction.

FIG. 16. Analysis of lentivirus transduction efficiency on indicator cells. Complexation of lentivirus (MOI 20), with various concentrations of NV00100028. Complexation occurred for various incubation times before adding lentivirus to HEK293T indicator cells. N=4

FIG. 17. Analysis of lentivirus transduction efficiency on indicator cells. Complexation of lentivirus (MOI 20), with various concentrations of NV00100026-28. Complexation occurred for various incubation times before adding lentivirus to HEK293T indicator cells. N=4

FIG. 18. Analysis of lentivirus transduction efficiency on indicator cells. Complexation of lentivirus (MOI 20), with various concentrations of NV0010032. Complexation occurred for various incubation times before adding lentivirus to HEK293T indicator cells. N=4

FIG. 19. Analysis of lentivirus transduction efficiency on indicator cells. Complexation of lentivirus (MOI 20), with various concentrations of NV0010033. Complexation occurred for various incubation times before adding lentivirus to HEK293T indicator cells. N=4

FIG. 20. Transduction efficiency of lentivirus (MOI 20) on indicator cells. Lentivirus was complexed with various batches of Nuvec®, at various concentrations for 10 min before addition to HEK293T indicator cells. GFP expression was measured via flow cytometry. N=4

FIG. 21. Transduction efficiency of lentivirus (MOI 20) on indicator cells. Lentivirus was complexed with various batches of Nuvec®, at various concentrations for 20 min before addition to HEK293T indicator cells. GFP expression was measured via flow cytometry. N=4

FIG. 22. Transduction efficiency of lentivirus (MOI 20) on indicator cells. Lentivirus was complexed with various batches of Nuvec®, at various concentrations for 30 min before addition to HEK293T indicator cells. GFP expression was measured via flow cytometry. N=4

FIG. 23. Average cell viability after transduction of lentivirus (MOI 20) with various concentrations of Nuvec® and various incubation times. N=2.

FIG. 24. Virus transduction of indicator cells+/−polybrene. Transduced cells using lentivirus at various limiting dilutions, with or without 5 μg/ml of polybrene. See Table 7.

FIG. 25 shows SEM images of the nanoparticles.

### EXAMPLES

### Example 1—Synthesis of Ram-SNPs

Resorcinol (0.4 g) and formaldehyde (37 wt %, 0.56 mL) were added to the solution composed of ammonia aqueous solution (28 wt %, 12 mL), deionized water (40 mL) and ethanol (280 mL). The mixture was stirred for 8 h at room temperature (−25° C.), then 2.4 mL of tetraethylorthosilicate (TEOS) was added to the solution and stirred for 8 minutes before the second addition of resorcinol (1.6 g) and formaldehyde (37 wt %, 2.24 mL). The mixture was stirred for 2 h at room temperature, and then collected by centrifugation at 4700 rpm (3877 rcf) for 5 min, washed with ethanol and dried at 50° C. overnight. Finally, Ram-SNPs were collected by calcination at 550° C. for 5 h in air.

### Example 2—Synthesis of PEI Modified RNPs

0.03 gram of Ram-SNPs fabricated above were dispersed into 10 mL water under ultrasonication (ensure the no obvious bulk particles sediment in the solution). 0.213 mL of 3-(Trihydroxysilyl)propyl methylphosphonate (HTPMP, 50 wt % in water) was added into another 10 mL of water, and then the 10 mL of nanoparticle solution mixed with HTPMP solution, and t stirred at 40° C. for 2 h. The phosphonate modified silica nanoparticles were collected by centrifugation at 12000 rpm (17,420 rcf) for 5 min, and washed by water once. These nanoparticles were then directly re-dispersed into 5 mL carbonate-bicarbonate buffer solution (pH=9.6) by ultrasonication.

Polyethylenimine (PEI, branched, mean MW of 10 k, Alfar Aesar) was dissolved into 10 mL of carbonate-bicarbonate buffer under ultrasonication. Then the particle solution and PEI solutions were mixed together and stirred at room temperature for 4 h. The PEI loaded nanoparticles were collected by centrifugation at 12000 rpm for 5 min, and then washed by water once. The nanoparticles were then resuspended into 3 mL of water, and frozen under liquid nitrogen for 30 min, followed by drying at freeze-dryer for 2 days. The thoroughly dried particles were stored in the fridge in a desiccator and are hereinafter referred to as “Nuvec®”.

### Example 3—Cell Viability

6×10⁵ HEK293T cells were seeded in Dulbecco's Modified Eagle Medium (DMEM), supplemented with 10% fetal bovine serum (FBS) and 1% Penicillin Streptomycin (hereinafter termed “complete medium”) and incubated at 37° C., 5% CO₂ overnight.

Cells were treated with various concentrations of Nuvec ranging from 1 μg to 80 μg/0.5 ml. Treated cells were incubated at 37° C., 5% CO₂ for either 4 h, 24 h or 48 h before replacing with fresh complete medium. Cell viability was measured every 24 h post medium change for 72 h via a trypan blue exclusion assay. A Countess™ automated cell counter was used according to manufacturer's instructions.

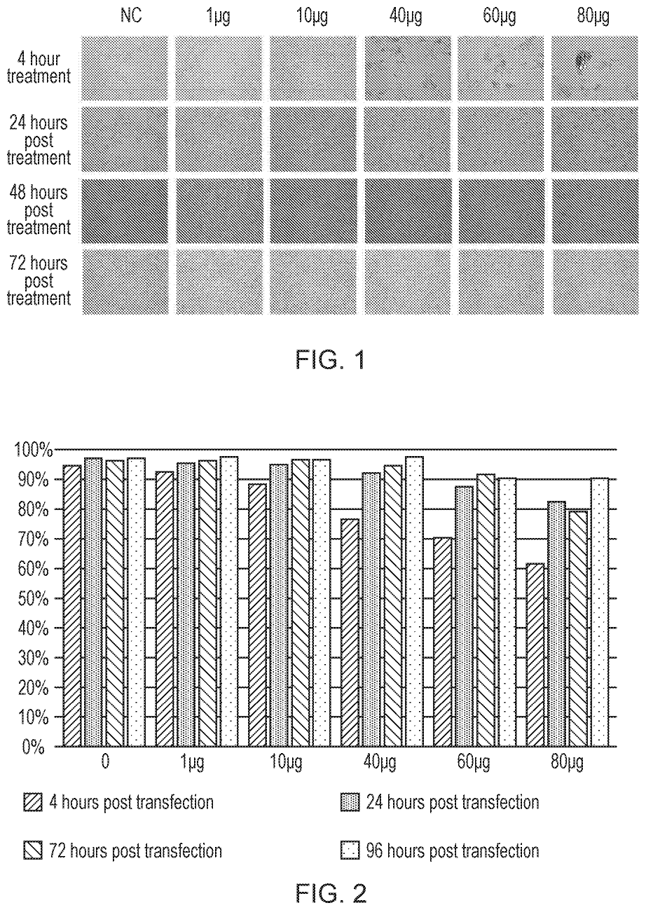

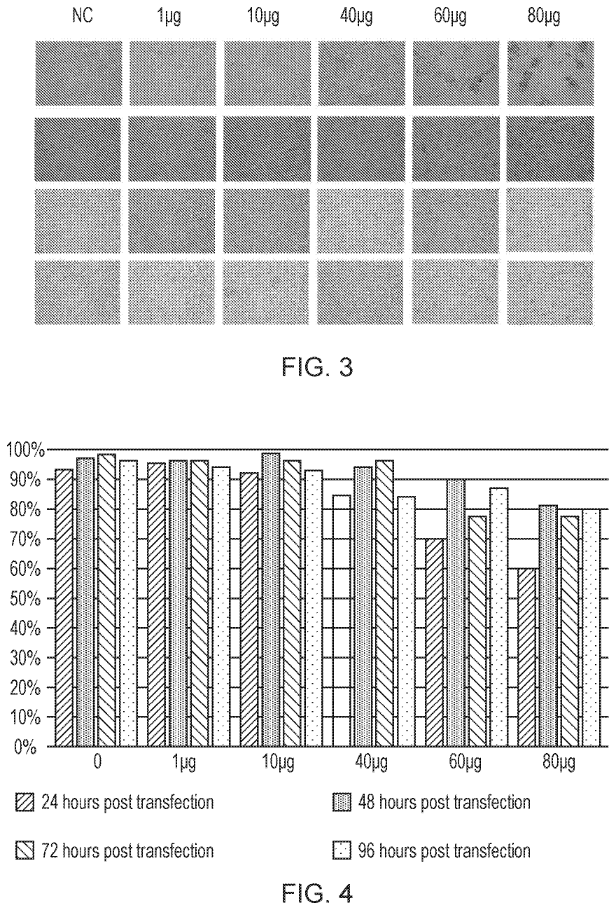

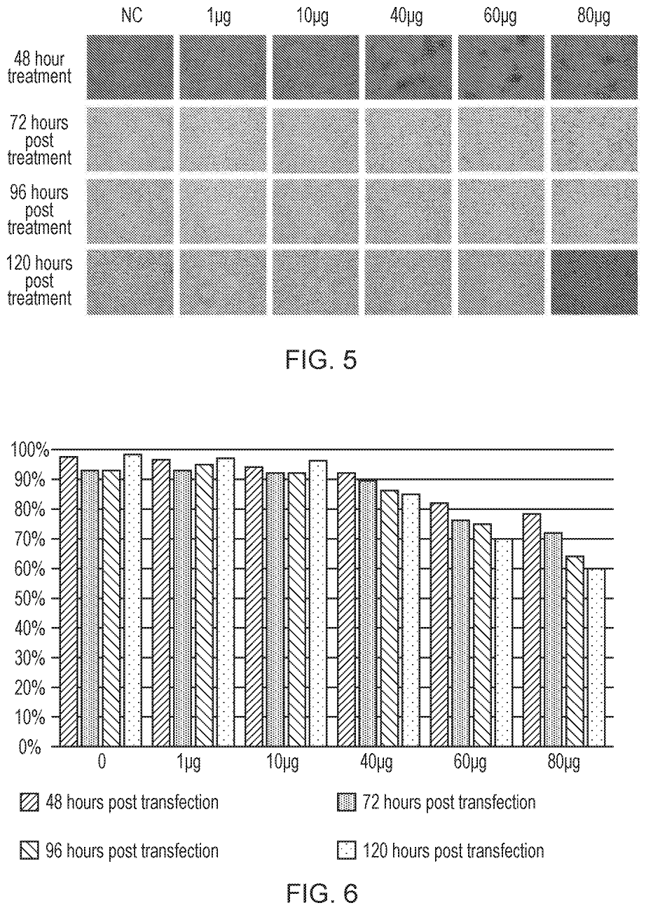

Cell viability analysis following a 4 h incubation with Nuvec is provided in FIGS. 1 and 2. Cell viability analysis following a 24 h incubation with Nuvec is provided in FIGS. 3 and 4. Cell viability analysis following a 48 h treatment with Nuvec is provided in FIGS. 5 and 6.

### Example 4— Single Plasmid Transfection Using Nuvec

The transfection efficacy of Nuvec was evaluated in HEK293T cells by assessing delivery efficiency of a plasmid DNA encoding green fluorescent protein (pDNA-EGFP).

Fresh HEK293T cells were seeded into 12 well plates at a density of 1.2×10⁵ cells per well in complete medium and incubated at 37° C., 5% CO₂ overnight to achieve 70-90% confluency.

Various amounts of Nuvec, ranging from 1-80 μg per well, were treated to minimise bioburden by resuspending the particles in at least 5 volumes of 70% ethanol, mixing, centrifuging briefly to collect the particles, then removing the clear supernatant. The suspension of Nuvec in PBS was then prepared by adding 50 μl sterile PBS to each well and sonicating using a bath sonicator with an output of at least 120 W until a homogeneous suspension was achieved (up to 30 min). Any fast-settling clumps during the sonication procedure were dispersed by pipetting.

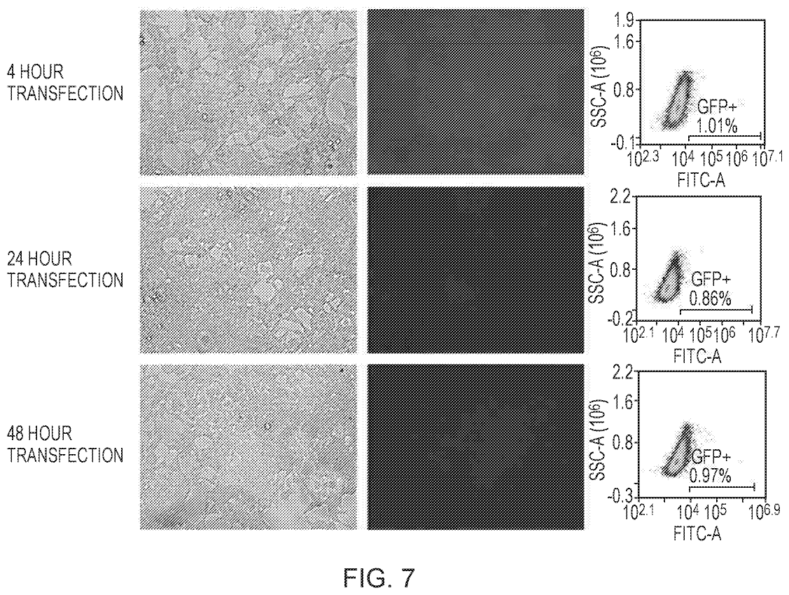

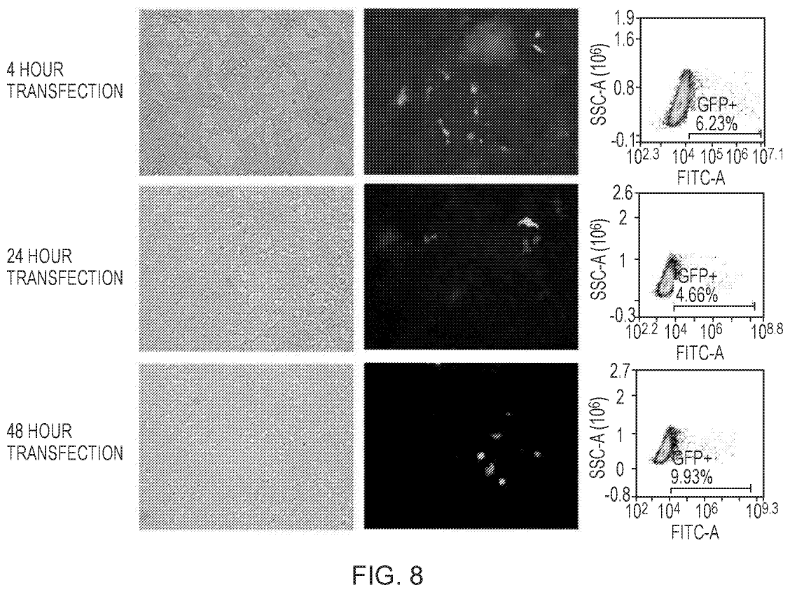

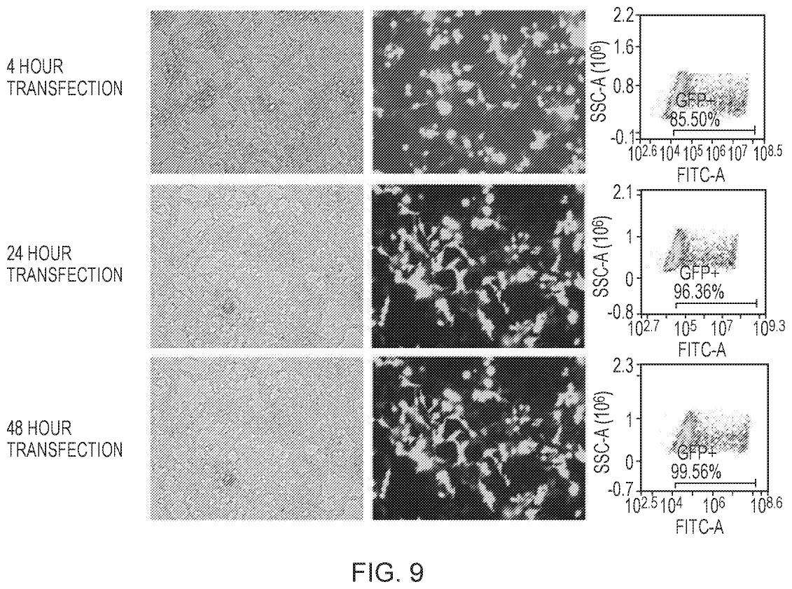

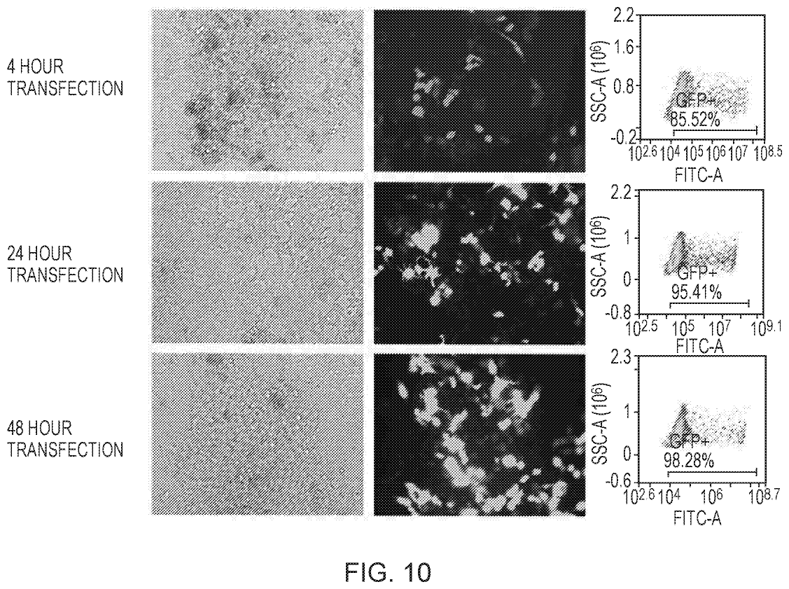

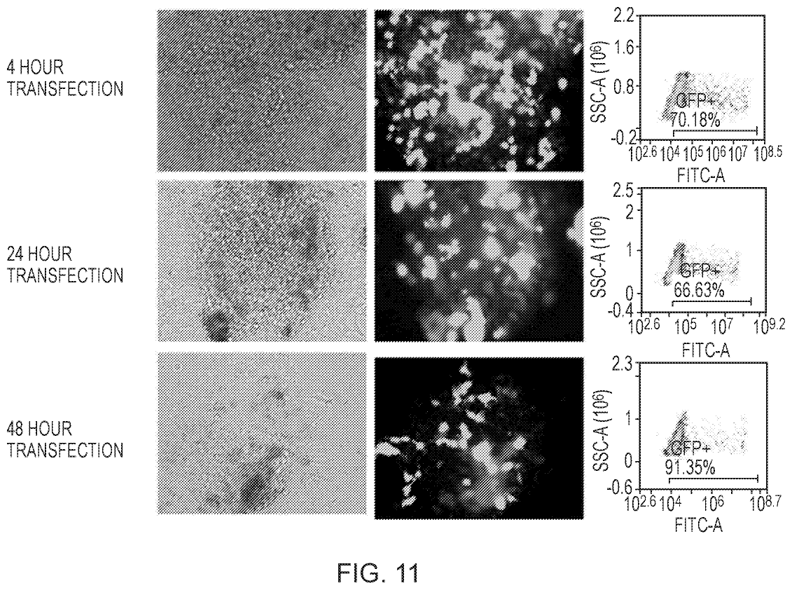

The various concentrations of Nuvec in PBS, ranging from 1 μg to 80 μg/0.5 ml, were complexed with a total of 1 μg of plasmid DNA for 30 min at room temperature. After complexation, the pDNA loaded Nuvec was then suspended into 1 ml of complete medium and used to replace the cell culture medium. Cell were incubated at 37° C., 5% CO₂ for 4 h, 24 h or 48 h, before replacing with fresh complete medium. Cells were analysed at 72 h post medium change by flow cytometry and confocal microscopy to determine the green fluorescent protein expression in cells. Experiments were performed in triplicate for each group. Results are provided in FIGS. 7-11.

### Example 5—Production of Lentivirus

1.5×10⁷ fresh HEK293T cells were seeded in a T175 flask in complete medium and incubated at 37° C., 5% CO₂ overnight to achieve confluency.

A total of 5 μg plasmid DNA (eGFP, pCMVR8.74 and pMD2.G) at a ratio of 4:3:1 was complexed with polybrene for 20 min in serum free medium (Opti-MEM). After complexation, the pDNA loaded transfection reagent was suspended in 1 ml complete medium and used to replace the cell culture medium. Cells were incubated at 37° C., 5% CO₂ for 24 h before replacing with fresh complete medium. Supernatant was harvested every 24 h for 72 h post replacement. Conditioned medium was filtered through 0.45 μM filters to remove cell debris and stored at 4° C. for future use.

Conditioned medium was concentrated via ultracentrifugation at 23,000 rpm at 4° C. The supernatant was discarded and the pellet air dried for 10 min. The pellet was resuspended in 200 μl serum free medium and incubated on ice for 1 h. The resuspended viral pellet was aliquoted and stored at −80° C. for future use.

### Example 6— Lentivirus Titration

Fresh HEK293T cells were seeded into 12 well plates at a density of 2×10⁵ cells/well in complete medium and incubated at 37° C., 5% CO₂ overnight.

Serial dilutions of virus were prepared and incubated in complete medium with 5 μg/ml polybrene for 20 min at room temperature. The virus polybrene mixture was then suspended into 1 ml complete medium and used to replace the cell culture medium. One well of cells was left as untreated and one well of cells were counted to determine the number of cells present for infection.

Cells were incubated at 37° C., 5% CO₂ for 24 h before replacing with fresh complete medium. Cells were analysed at 48 h post medium replacement by flow cytometry analysis of GFP expression. Only samples expressing 1-30% GFP expression were analysed as accurate representations of viral titre. Virus titre was calculated at each dilution point, as shown below, and averaged for overall average titre.

Titre(TU/ml)=((Cell count*(Percentage GFP expression/100))/Volume)*DF

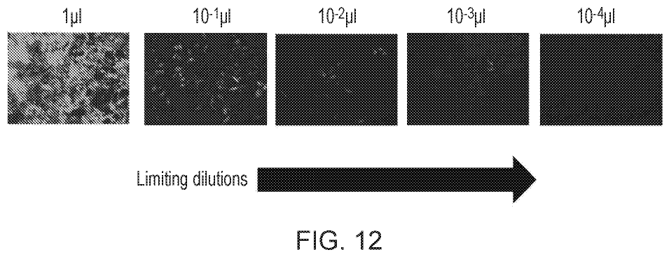

TU/ml is transduction units per ml. DF is the dilution factor. FIG. 12 shows images of GFP expression at limiting dilutions. A titre of 1.18×10⁹TU/ml was calculated.

### Example 7— Triple Plasmid Transfection of Cells

Fresh HEK293T cells were seeded into 6 well plates at a density of 6×10⁵ cells per well in complete medium and incubated at 37° C., 5% CO₂ overnight to achieve confluency.

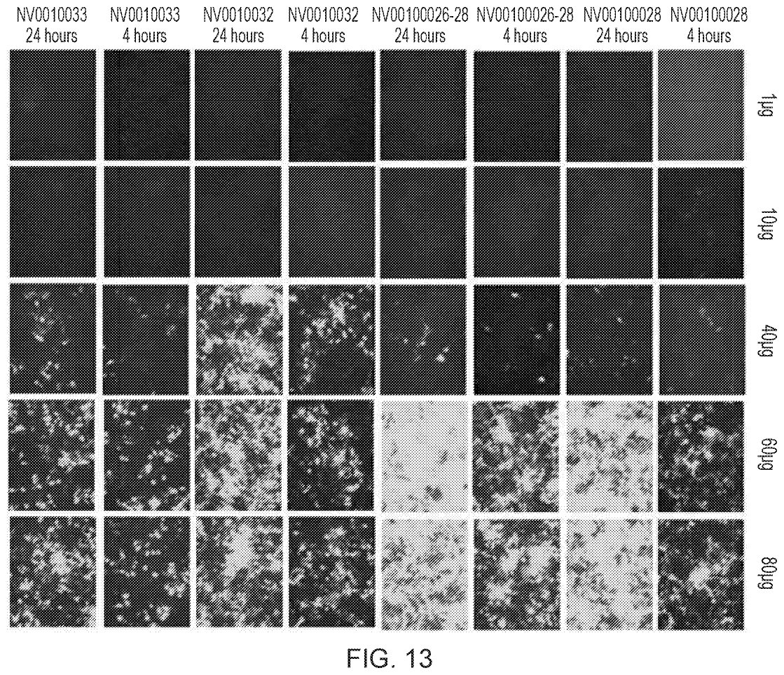

Various concentrations of Nuvec, ranging from 1 μg to 80 μg/0.5 ml, were complexed with a total of 5 μg of plasmid DNA (eGFP, pCMVR8.74 and pMD2.G) at a ratio of 4:3:1 for 30 min in serum free medium. Different batches of Nuvec were also tested. After complexation, the pDNA loaded Nuvec was suspended in 1 ml complete medium and used to replace the cell culture medium. Cells were incubated at 37° C., 5% CO₂ for either 4 h or 24 h before replacing with fresh complete medium. Supernatant was harvested every 24 h for 72 h post medium replacement. Cells were analysed at 72 h post-transfection for GFP expression using fluorescence microscopy. Conditioned medium was centrifuged at 1500 rpm for 5 min with the supernatant collected and stored at 4° C. for future use. FIG. 13 shows transfected cells 72 h post-transfection.

Transfection experiments were repeated a total of three times for NV00100028, NV00100026-28, NV0010032 and NV0010033 as well as 4 hour and 24 hour transfections. The titres generated (TU/ml) were calculated and are shown in the table below.

TABLE 1
Transfection Experiments (all values are “×10⁴”).

| | 1 μg | | | 10 μg | | | 40 μg | | | 60 μg | | | 80 μg | | |
|---|---|---|---|---|---|---|---|---|---|---|---|---|---|---|---|
| N | 1 | 2 | 3 | 1 | 2 | 3 | 1 | 2 | 3 | 1 | 2 | 3 | 1 | 2 | 3 |
| NV00100028 | 0 | 0 | 0 | 0 | 0 | 0 | 3.02 | 4.21 | 3.25 | 61.6 | 8.48 | 4.65 | 58.1 | 131 | 31.1 |
| 4 hours | | | | | | | | | | | | | | | |
| NV00100028 | 0 | 0 | 0 | 0 | 0 | 0 | 06.7 | 3.97 | 5.83 | 149 | 13.3 | 8.72 | 104 | 221 | 32.4 |
| 24 hours | | | | | | | | | | | | | | | |
| NV00100026-28 | 0 | 0 | 0 | 0 | 0 | 0 | 5.36 | 3.65 | 0 | 130 | 4.51 | 0 | 64.0 | 0 | 0 |
| 4 hours | | | | | | | | | | | | | | | |
| NV00100026-28 | 0 | 0 | 0 | 0 | 0 | 0 | 2.97 | 2.81 | 0 | 213 | 3.38 | 0 | 124 | 0 | 0 |
| 24 hours | | | | | | | | | | | | | | | |
| NV0010032 | 0 | 0 | 0 | 0 | 0 | 0 | 22.3 | 0 | 0 | 56.1 | 2.92 | 2.75 | 68.2 | 12.6 | N/A |
| 4 hours | | | | | | | | | | | | | | | |
| NV0010032 | 0 | 0 | 0 | 0 | 0 | 0 | 17.9 | 0 | 0 | 52.5 | 4.70 | 4.35 | 52.2 | 16.3 | 6.88 |
| 24 hours | | | | | | | | | | | | | | | |
| NV0010033 | 0 | 0 | 0 | 0 | 0 | 0 | 3.02 | 0 | 0 | 0 | 0 | 0 | 0 | 0 | 0 |
| 4 hours | | | | | | | | | | | | | | | |
| NV0010033 | 0 | 0 | 0 | 0 | 0 | 0 | 0.67 | 0 | 0 | 4.30 | 4.70 | N/A | 15.1 | 16.3 | 0 |
| 24 hours | | | | | | | | | | | | | | | |

### Example 8—Transduction of Cells with Conditioned Medium

Fresh HEK293T cells were seeded into 12 well plates at a density of 2×10⁵ cells/well and incubated at 37° C., 5% CO₂ overnight to achieve confluency.

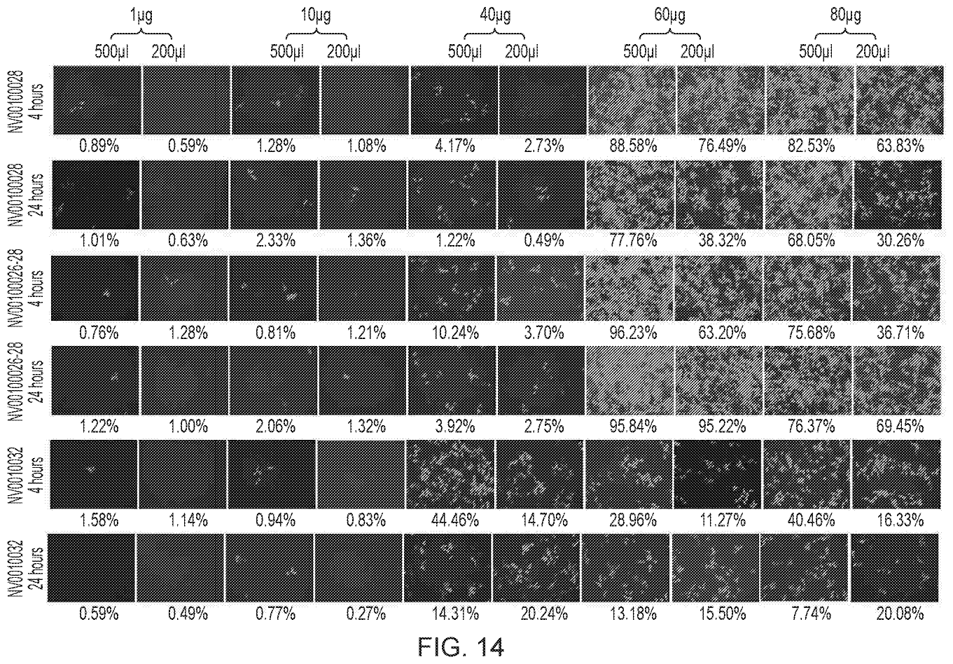

Cells were transduced with 500p1 or 200p1 conditioned medium collected from cells transfected in Example 7. Cells were incubated at 37° C., 5% CO₂ for 24 h before replacing with fresh complete medium. Cells were analysed at 72 h post-transduction for GFP expression using fluorescence microscopy and flow cytometry. The results are provided in FIG. 14.

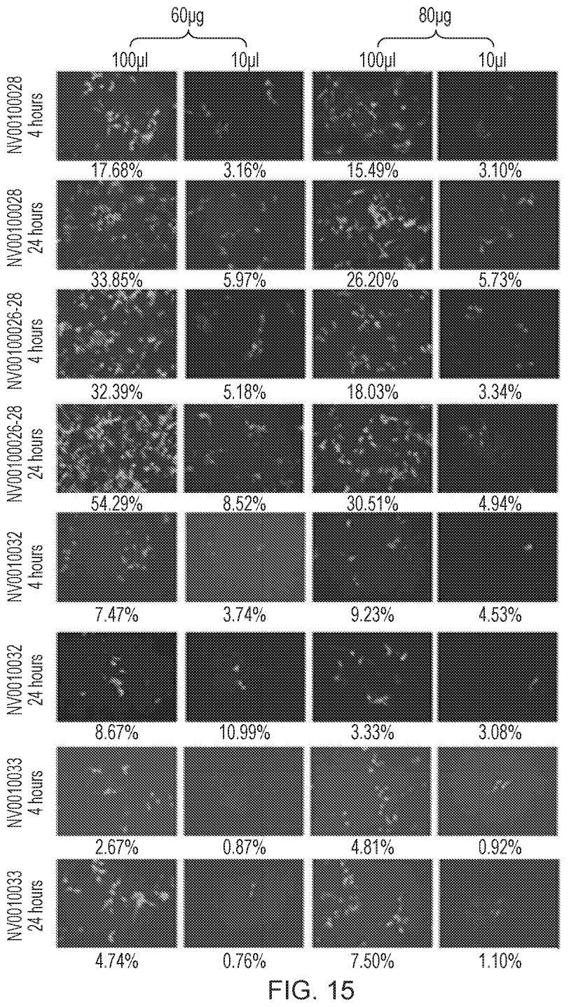

Fresh HEK293T cells were transduced with 100p1 or 10p1 conditioned medium collected from cells transfected in Example 7. Cells were incubated at 37° C., 5% CO₂ for 24 h before replacing with fresh complete medium. Cells were analysed at 72 h post transduction for GFP expression using fluorescence microscopy and flow cytometry. The results are provided in FIG. 15.

### Example 9—Transduction with Lentivirus

Fresh HEK293T cells were seeded into 12 well plates at a density of 2×10⁵ cells/well in complete medium and incubated at 37° C., 5% CO₂ overnight to achieve confluency.

Various concentrations of Nuvec, ranging from 1 μg to 80 μg/0.5 ml, were complexed with lentivirus carrying a GFP transgene (MOI 20) for 10 min, 20 min or 30 min at room temperature. The lentivirus was made according to Example 5. Different batches of Nuvec® were tested.

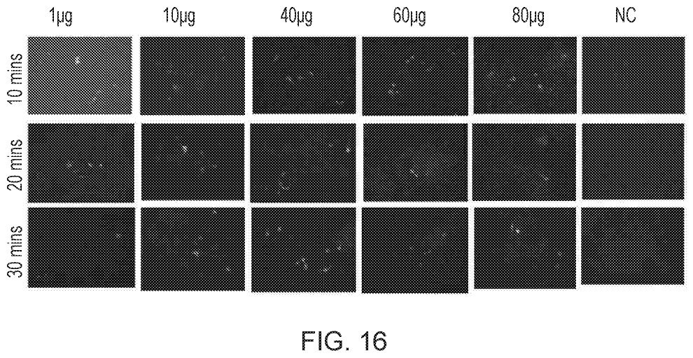

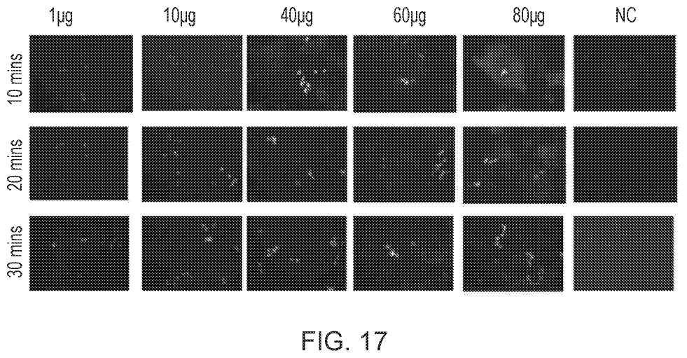

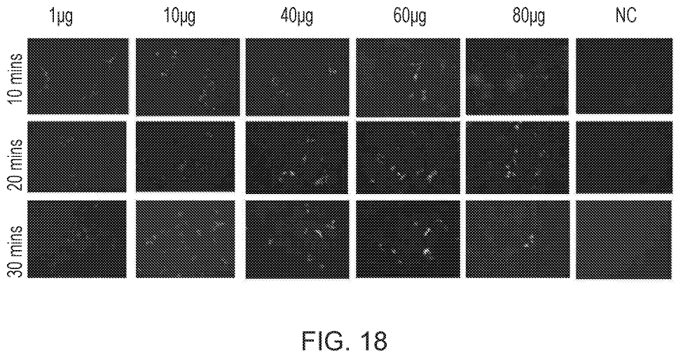

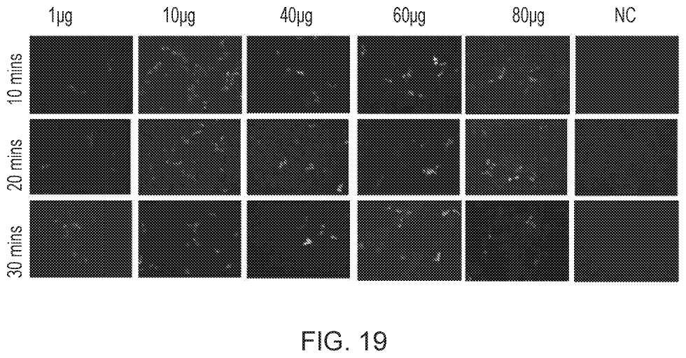

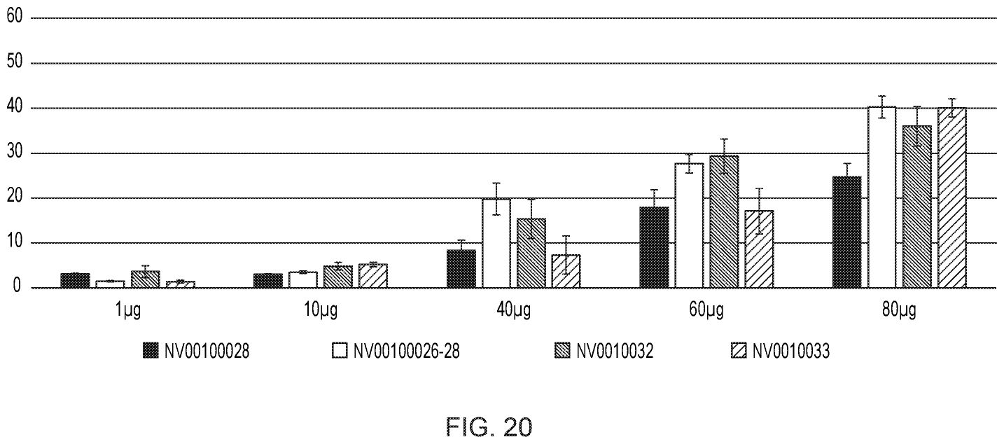

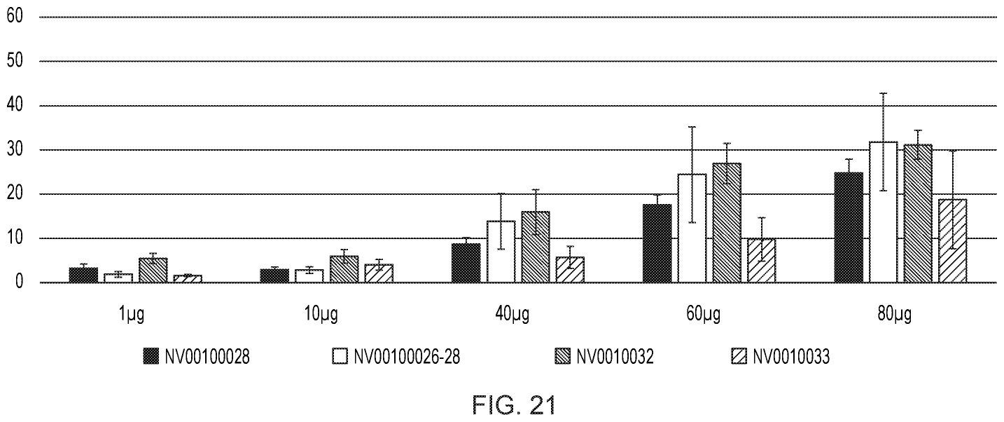

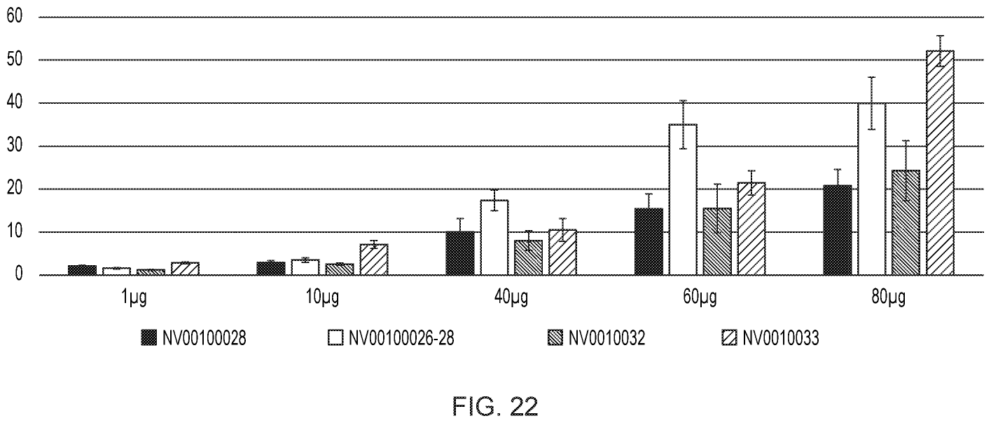

After complexation, the lentivirus and Nuvec mixture was suspended in 1 ml complete medium and used to replace the cell culture medium. Cells were incubated at 37° C., 5% CO₂ for 24 h before replacing with fresh complete medium. Cells were analysed at 72 h post-transduction for GFP expression using fluorescence microscopy and flow cytometry. FIGS. 16-22 show GFP expression of transduced cells using different batches of Nuvec.

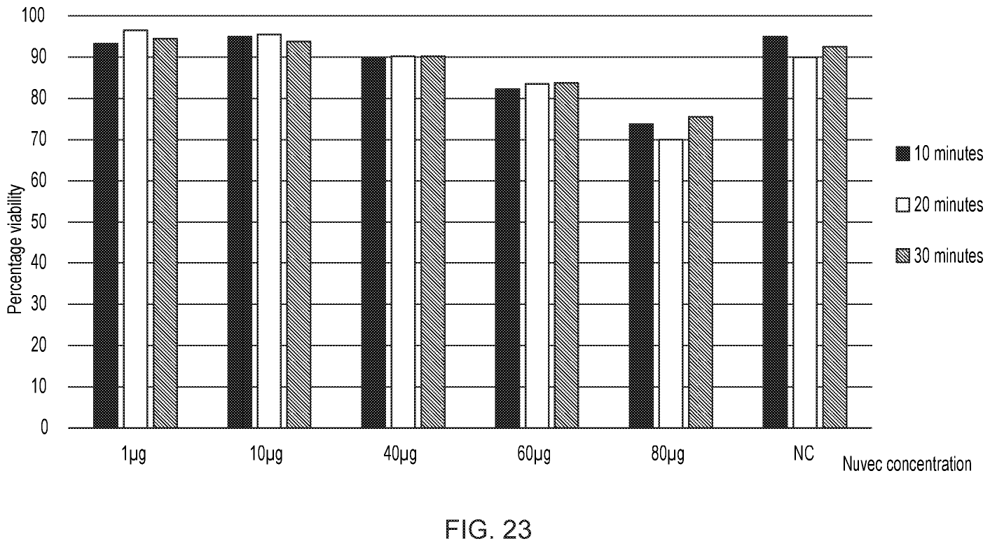

Viability of transduced cells was analysed using a trypan blue exclusion assay. The results are provided in FIG. 23.

### Example 10—Comparison with Standard Transduction Protocol

Fresh HEK293T cells were seeded into 12 well plates at a density of 2×10⁵ cells/well in complete medium and incubated at 37° C., 5% CO₂ overnight to achieve confluency.

Various limiting dilutions of lentivirus carrying a GFP transgene (MOI 20) was complexed with 5 μg/ml polybrene for 20 mins at room temperature. Lentivirus incubated in the absence of polybrene was used as a control.

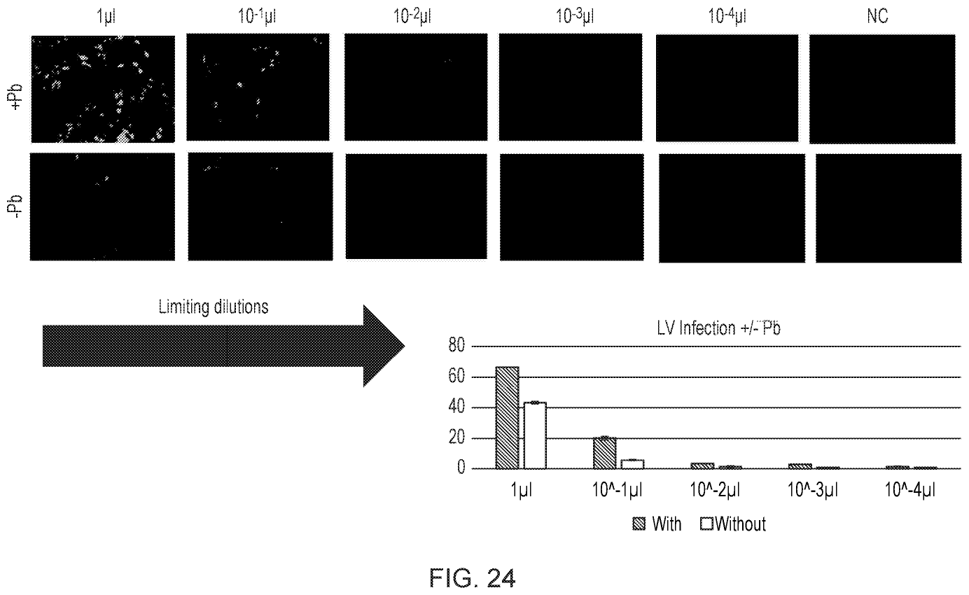

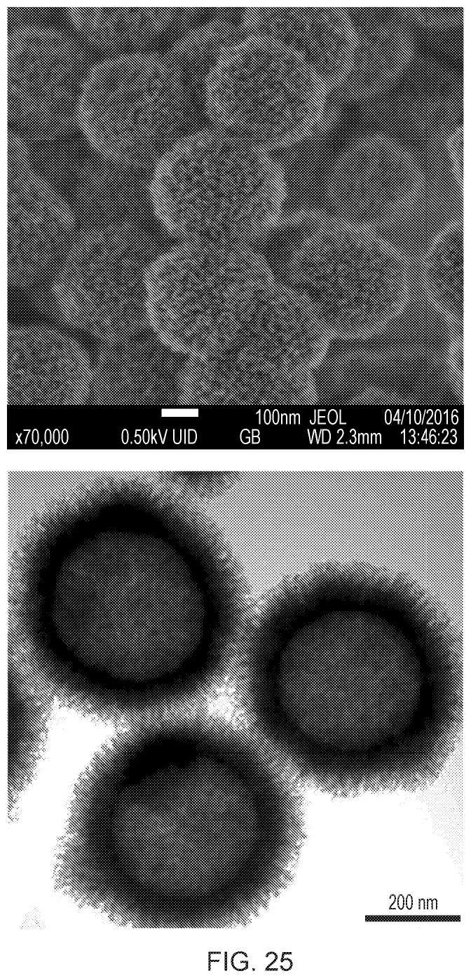

After complexation, the lentivirus and polybrene mixture was suspended in 1 ml complete medium and used to replace the cell culture medium. Cells were incubated at 37° C., 5% CO₂ for 24 h before replacing with fresh complete medium. Cells were analysed at 72 h post-transduction for GFP expression using fluorescence microscopy and flow cytometry. FIG. 24 shows GFP expression of transduced cells.

A summary of the transduction efficiency is provided in the table below.

TABLE 2
Transduction Efficiency Information.

| | Nuvec ® concentration/μg | | | | |
|---|---|---|---|---|---|
| Batch | 1 μg | 10 μg | 40 μg | 60 μg | 80 μg |
| 10 minutes | | | | | |
| NV00100028 | 3.14 ± 0.21 | 3.02 ± 0.15 | 8.36 ± 2.31 | 17.92 ± 3.90 | 24.76 ± 2.99 |
| NV00100026-28 | 1.51 ± 0.17 | 3.20 ± 0.26 | 19.81 ± 3.61 | 27.71 ± 2.04 | 40.33 ± 2.46 |
| NV0010032 | 3.65 ± 1.32 | 4.87 ± 0.88 | 15.35 ± 4.30 | 29.38 ± 3.80 | 36.03 ± 4.37 |
| NV0010033 | 1.43 ± 0.33 | 5.22 ± 0.51 | 7.30 ± 4.24 | 17.11 ± 5.04 | 40.09 ± 2.01 |
| 20 minutes | | | | | |
| NV00100028 | 3.32 ± 0.96 | 2.98 ± 0.58 | 8.77 ± 1.40 | 17.55 ± 2.33 | 24.81 ± 3.22 |
| NV00100026-28 | 1.90 ± 0.64 | 2.88 ± 0.73 | 13.86 ± 6.30 | 24.44 ± 10.83 | 31.83 ± 11.04 |
| NV0010032 | 5.47 ± 1.15 | 5.97 ± 1.52 | 15.99 ± 5.06 | 26.97 ± 4.56 | 31.20 ± 3.24 |
| NV0010033 | 1.64 ± 0.24 | 4.08 ± 1.21 | 5.73 ± 2.51 | 9.80 ± 4.93 | 18.76 ± 11.06 |
| 30 minutes | | | | | |
| NV00100028 | 2.11 ± 0.23 | 2.91 ± 0.42 | 10.02 ± 3.13 | 15.39 ± 3.46 | 20.77 ± 3.78 |
| NV00100026-28 | 1.56 ± 0.14 | 3.51 ± 0.52 | 17.34 ± 2.42 | 35.03 ± 5.57 | 39.97 ± 6.05 |
| NV0010032 | 1.17 ± 0.14 | 2.55 ± 0.25 | 7.99 ± 2.27 | 15.20 ± 5.63 | 24.30 ± 7.02 |
| NV0010033 | 2.81 ± 0.23 | 7.12 ± 0.88 | 10.47 ± 2.66 | 21.42 ± 2.80 | 52.11 ± 3.55 |
| +Polybrene | 2.61 ± 0.46 | | −Polybrene | 1.40 ± 0.40 | |

TABLE 3
Viability data of cells treated in accordance with FIG. 2.

| | Viability 4 | Viability 24 | Viability 48 | Viability 72 |
|---|---|---|---|---|
| | hours post | hours post | hours post | hours post |
| Concentration | treatment | treatment | treatment | treatment |
| 0 | 95% | 98% | 97% | 98% |
| 1 μg | 93% | 96% | 97% | 98% |
| 10 μg | 89% | 96% | 97% | 97% |
| 40 μg | 77% | 93% | 95% | 98% |
| 60 μg | 71% | 88% | 92% | 91% |
| 80 μg | 62% | 83% | 80% | 91% |

TABLE 4
Viability data of cells treated in accordance with FIG. 4.

| | Viability 24 | Viability 48 | Viability 72 | Viability 96 |
|---|---|---|---|---|
| | hours post | hours post | hours post | hours post |
| Concentration | treatment | treatment | treatment | treatment |
| 0 | 93% | 97% | 98% | 96% |
| 1 μg | 95% | 96% | 96% | 94% |
| 10 μg | 92% | 98% | 96% | 93% |
| 40 μg | 84% | 94% | 96% | 84% |
| 60 μg | 70% | 90% | 77% | 87% |
| 80 μg | 60% | 81% | 77% | 80% |

TABLE 5
Viability data of cells treated in accordance with FIG. 6.

| | Viability 48 | Viability 72 | Viability 96 | Viability 120 |
|---|---|---|---|---|
| | hours post | hours post | hours post | hours post |
| Concentration | treatment | treatment | treatment | treatment |
| 0 | 97% | 93% | 93% | 98% |
| 1 μg | 96% | 93% | 95% | 97% |
| 10 μg | 94% | 92% | 92% | 96% |
| 40 μg | 92% | 90% | 86% | 97% |
| 60 μg | 82% | 76% | 75% | 95% |
| 80 μg | 78% | 72% | 64% | 60% |

TABLE 6
Dilutions as shown in FIG. 12.

| Dilution | % GFP | Titre/ml × 10⁸ |
|---|---|---|
| 1 | 84.12 | 4.33 |
| 1 | 83.38 | 4.29 |
| −1 | 27.57 | 14.2 |
| −1 | 25.47 | 13.1 |
| −2 | 3.36 | 17.3 |
| −2 | 3.4 | 17.5 |
| −3 | 0.81 | 41.7 |
| −3 | 0.91 | 46.9 |
| −4 | 1.03 | 530 |
| −4 | 0.59 | 304 |

TABLE 7
Dilutions as shown in FIG. 24.

| | | 1 × | 1 × | 1 × | 1 × |
|---|---|---|---|---|---|
| Dilution | 1 μl | 10⁻¹ μl | 10⁻² μl | 10⁻³ μl | 10⁻⁴ μl |
| With Pb | 66 ± 0.2 | 20 ± 1 | 3 ± 0.1 | 3 ± 0.1 | 1 ± 0.3 |
| Without Pb | 43 ± 0.8 | 6 ± 0.4 | 1 ± 0.4 | 2 ± 0.26 | 0.7 ± 0.1 |

1. A composition comprising:
an inorganic mesoporous nanoparticle comprising silica; and
one or more delivery components;
wherein the nanoparticle comprises projections thereon;
wherein the nanoparticle has a diameter in the range 50 nm to 3000 nm;
wherein the inorganic nanoparticle is at least partially coated with a transfection agent; and
wherein the one or more delivery components comprises a viral vector.

2. The composition according to claim 1, wherein the viral vector is an adenoviral vector, an ademo-associated viral vector, or a retroviral vector.

3. The composition according to claim 1, wherein the nanoparticle comprises:
a shell comprising silica;
a hollow core with a volume defined by the inner surface of the shell; and
a plurality of projections comprising silica disposed on the exterior of the shell.

4. (canceled)

5. The composition according to claim 1, wherein the nanoparticle is hollow.

6. The composition according to claim 1, wherein the nanoparticle is rambutan-like or morningstar-like.

7. The composition according to claim 1, wherein the projections comprise fingers or spikes.

8. The composition according to claim 1, wherein the transfection agent is a cationic polymer.

9. The composition according to claim 8, wherein the transfection agent is a polyalkylimine, chitosan, polylysine, DEAE-dextran, polybrene, or polyamidoamine (PAMAM) dendrimer.

10. The composition according to claim 1, wherein the nanoparticle is at least partially coated with the one or more delivery components.

11. The composition according to claim 1, wherein the nanoparticle comprises at least two delivery components.

12-14. (canceled)

15. The composition according to claim 1, further comprising a cell.

16. The composition according to claim 15, wherein the cell is a CAR-T cell or a TCR-T cell.

17-20. (canceled)

21. A method of treating a disease in a patient, comprising administering the composition according to claim 1 to the patient.

22. A method of manufacturing the composition according to claim 1, the method comprising the step of:
i) mixing an inorganic mesoporous nanoparticle comprising silica and having projections thereon with one or more delivery components; wherein the diameter of the nanoparticles are in range of from 50 nm to 3000 nm; and wherein the one or more delivery components comprises a viral vector.

23. A method of transfecting or transducing a cell comprising the steps of:
(i) providing a composition according to claim 1; and
(ii) incubating the composition with a cell.

24. (canceled)

25. (canceled)

26. The composition according to claim 2, wherein the viral vector is an adenoviral vector or a lentiviral vector.

27. The composition according to claim 3, wherein the projections are integral with the shell.

28. The composition according to claim 3, wherein the projections extend radially outwards from the shell.

29. The composition according to claim 1, wherein the projections have a length of 5 nm to 1000 nm, from 10 nm to 200 nm, or from 50 nm to 150 nm.

30. The composition according to claim 9, wherein the transfection agent is a polyalkylimine or a polyethylimine (PEI).

31. The method of claim 21, wherein the disease is a genetic disorder, cancer, infection, or autoimmune disease.
